(19) United States
(12) Patent Application Publication
(10) Pub. No.: US 2004/0181129 A1
(43) Pub. Date: Sep. 16, 2004

(54) Personal physical fitness diagnostic evaluation system & method

(76) Inventor: James C. Glasgow, Raleigh, NC (US)

Correspondence Address: JINAN GLASGOW, P O BOX 28539, RALEIGH, NC, 276118539

(21) Appl. No.: 10/799,383

(22) Filed: Mar. 12, 2004

Related U.S. Application Data
(60) Provisional application No. 60/455,098, filed on Mar. 14, 2003.

(51) Int. Cl. A61B 5/00

(52) U.S. Cl. 600/300; 128/920

(57) ABSTRACT

A system and method for facilitating personal physical fitness diagnostic evaluations including a handheld electronic device having input and output means is provided with formulas for calculating predetermined diagnostic physical fitness indicators, where a user inputs data and an output is generated therefrom for indicating the physical fitness of an individual based on predetermined parameters.

### CROSS-REFERENCE TO RELATED APPLICATIONS

This non-provisional utility patent application claims the benefit of a prior filed provisional application No. 60/455,098 filed Mar. 14, 2003, which is incorporated herein by reference in its entirety.

### BACKGROUND OF THE INVENTION

(1) Field of the Invention

The present invention relates generally to physical fitness and, more particularly, to a system and method for facilitating personal physical fitness diagnostic evaluations.

(2) Description of the Prior Art

The present invention is directed to a personal fitness diagnostic system and method for facilitating diagnostic evaluation for an individual. In a preferred embodiment, a handheld electronic device having input and output means is provided with formulas for calculating predetermined diagnostic physical fitness indicators, where a user inputs data and an output is generated therefrom for indicating the physical fitness of an individual based on predetermined parameters.

The personal training industry is beginning a new era. The general public is receiving more and more media hype about getting into shape to better enhance their lifestyles. How does an individual really know if they are “getting into shape”, “toning up”, or “putting on muscle”? Is feeling better really measurable? Stepping on the scales can be deceiving and frustrating too. Providing measurable results from an established baseline truly demonstrates effectiveness from any training program regardless of the client's goals. Providing clients a printable baseline demonstrates the beginning point and illustrates the cutting edge of technology with personal training. The key to client retention is to achieve results and to provide immediate feedback on the client's baseline fitness evaluation and ongoing improvements.

Prior art fitness evaluations commonly employs manual data gathering and calculation, which can be time-consuming and introduce opportunities for mistakes in data documentation or errors in calculation. While calculators or computers have been used in the past to aid in providing results from fitness evaluation and testing, these are generally limited in application because they are not usable at the site of the fitness testing and do not provide the mobility that a personal trainer requires in providing services to clients, particularly in a typical gym or fitness setting.

Thus, there remains a need for a lightweight, mobile, hand-held device for use in personal training having automatic calculation of fitness factors for use in an evaluation of an individual's physical fitness, both at a given instant and as it changes over time with respect to a benchmark or baseline, such as an initial screening or testing at the start of an exercise or fitness program, in particular where a personal trainer is administering the testing and involved in providing guidance, training, and oversight in the individual's fitness program.

### SUMMARY OF THE INVENTION

The present invention is directed to a hand-held device for use in personal training and fitness evaluation for automatically calculating predetermined, select factors relevant to an individual's fitness based upon user inputs.

In the preferred embodiment, the present invention provides for automatic calculation of select, predetermined factors relevant for an individual's fitness evaluation and testing and produces results in a printable format for providing a hard copy to the individual or for a file.

Preferably, a hand-held computer-type device is used to store the formulas in an executable format when data is input by the user for the automatic calculation of select, predetermined factors relevant for an individual's fitness evaluation and testing.

The present invention is further directed to a method for automatic calculation of select, predetermined factors relevant for an individual's fitness evaluation and testing.

Accordingly, one aspect of the present invention is to provide a hand-held computer-type device is used to store the formulas in an executable format when data is input by the user for the automatic calculation of select, predetermined factors relevant for an individual's fitness evaluation and testing.

Another aspect of the present invention is to provide a method for automatic calculation of select, predetermined factors relevant for an individual's fitness evaluation and testing including the steps of providing a hand-held device having a software program with formulas relevant for an individual's fitness evaluation and testing; a user inputting data relevant to the individual to be evaluated; the device automatically calculating select, predetermined factors; and the device outputting the factors in a readable and/or printable format.

These and other aspects of the present invention will become apparent to those skilled in the art after a reading of the following description of the preferred embodiment.

### BRIEF DESCRIPTION OF THE DRAWINGS

FIG. 1 shows a user interface view of input/outputs according to the system and method according to the present invention.

FIG. 2 shows another user interface view of input/outputs according to the system and method according to the present invention.

FIG. 3 shows another user interface view of input/outputs according to the system and method according to the present invention.

### DETAILED DESCRIPTION OF THE PREFERRED EMBODIMENTS

In the following description, it is to be understood that such terms as “forward,” “rearward,” “front,” “back,” “right,” “left,” “upwardly,” “downwardly,” and the like are words of convenience and are not to be construed as limiting terms.

Referring now to a preferred embodiment for the purpose of describing the invention, the following descriptions are not intended to limit the invention thereto.

The present invention, including a hand-held computer-type device is used to store the formulas in an executable format when data is input by the user for the automatic calculation of select, predetermined factors relevant for an individual's fitness evaluation and testing, has the capability to perform the health history questionnaire noting red flags that may limit prescription of program or fitness testing for the client, personal goals information concerning client as well as commitment goals, and determining baseline specs of client. The present invention also includes a hand-held computer-type device is used to store the formulas in an executable format when data is input by the user for the automatic calculation of select, predetermined factors relevant for an individual's fitness evaluation and testing. A software is also provided according to the present invention that may be run on the device or alternatively on a computer for providing the automatic calculation of select, predetermined factors and other user input factors relevant for an individual's personal fitness evaluation, testing, and tracking over time. Preferably, the software provides for information relating to at least one individual and preferably a multiplicity of individual's information to be stored in a database format for storing, retrieving, searching, and updating user information.

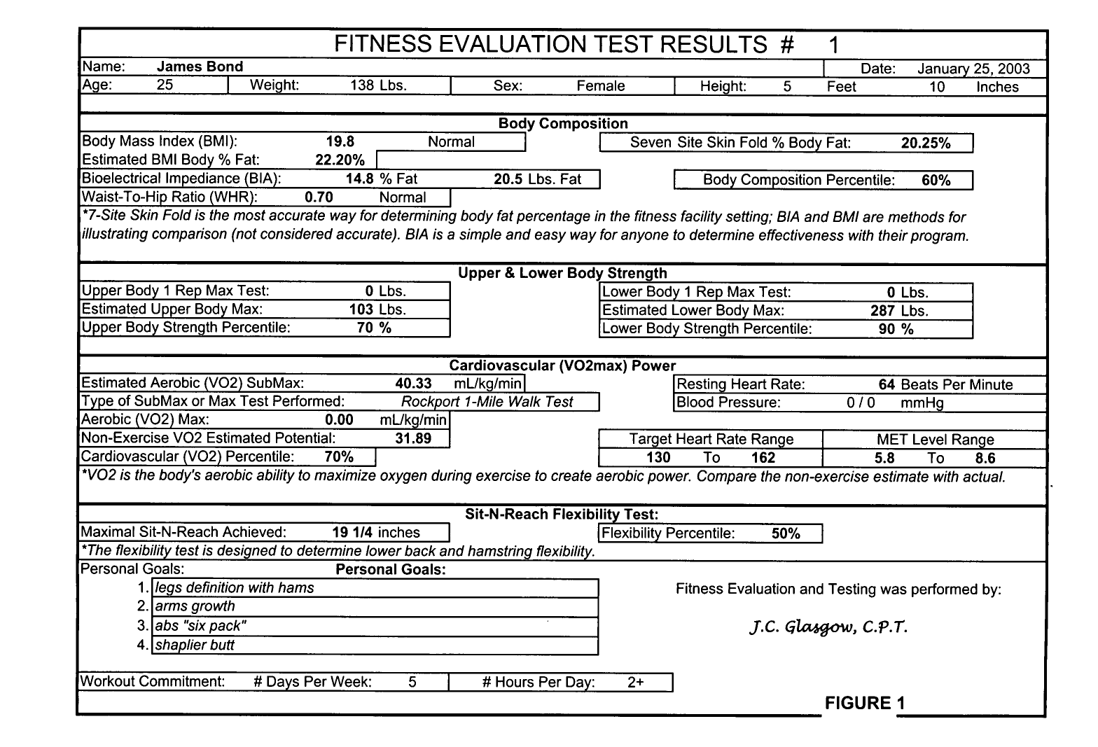

FIG. 1 shows a user interface view of input/outputs according to the system and method according to the present invention, specifically fitness evaluation test results outputs based upon inputs provided by a user, the inputs including but not limited to name, age, weight, sex, height, date, BMI, body fat percentage, upper and lower body strength, cardiovascular power, sit and reach flexibility test results, personal goals, and workout commitment based upon user interaction with the testee.

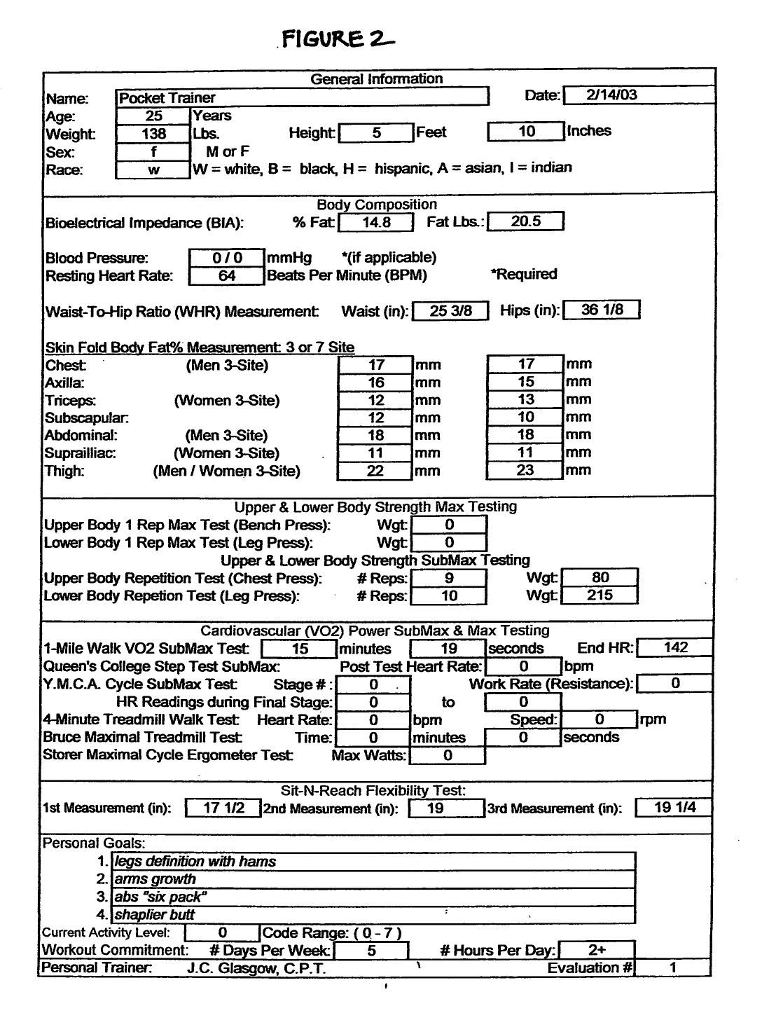

FIG. 2 shows another user interface view of input/outputs according to the system and method according to the present invention, namely general information, body composition, upper and lower body strength max testing, sit and reach flexibility test, personal trainer, and the like.

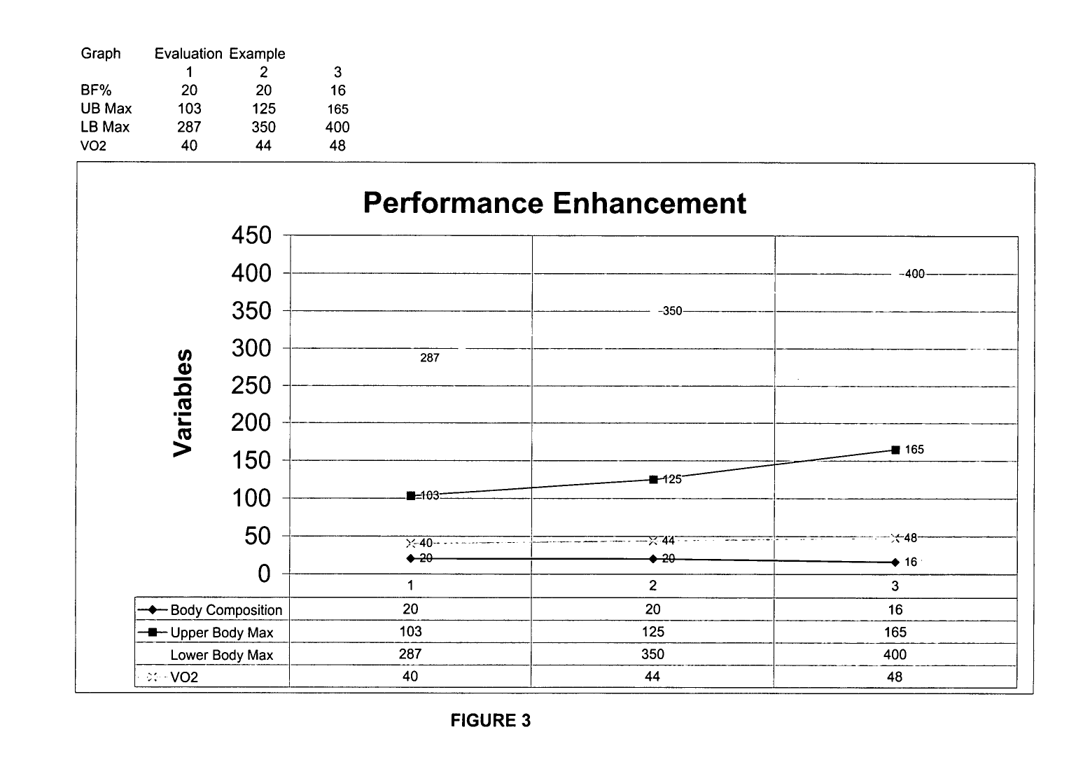

FIG. 3 shows another user interface view of input/outputs according to the system and method according to the present invention, namely performance enhancement based upon predetermined variables.

The device and software running thereon preferably capable of performing the following functions individually, collectively, and/or in combination: Differentiating between various information inputted during questionnaire that is pertinent in calculating proper equations based on specific tests performed but not limited to baseline data and test evaluation results. Determining proper calculation of specific equations regarding tests aid to determine and develop client's results. Providing explanations for test results in both common and scientific language. Functions include determining results based on data entered and obtained from client's fitness evaluation/test through calculations from equations and data base information. Some information is transposed from data entry. Capability to apply fitness evaluation results to resistance training and cardiovascular training programs as well as fitness evaluation test results. Descriptions of safe/proper testing protocols as well as functioning for effective exercises during resistance and cardio training. Basis for comparison between non-exercise/test results with actual accurate measurements performed during evaluation. Capability to determine resting heart rate, training heart rate, and blood pressure with ease to client. Ability to transport and record data throughout facility while testing of client is in progress. Maintain database of clients' records from evaluation to evaluation. Produce graphing capabilities to illustrate progress with clients from evaluation to evaluation and workout to workout. Produce printable hardcopies for clients' personal files. Compatibility to function with facilities' computer systems makes it fully operational without major upgrades. Multifunctional capability is easily understand and operate by personal trainers at facility. The present invention hardware is compact in size and simple Q & A format aids quick accurate results. Total operational access is simplified without mainframe computer on facility to print results. The addition of the present invention software modifies and enhances the detailed capabilities of the present invention hardware. Bottom line to perform the tasks of initial and on-going fitness evaluation/testing/program development by personal trainers or in-home individuals to easily gain accurate results as well as illustrate progress and provide information to maintain progress.

TABLE 1
Fitness Factors

```
Body Composition
  Transposes BIA for comparison w/Skin fold & BMI
  Calculates BMI
  Determines results of BMI calculation
  Calculates non-test estimate of body fat from BMI
  Choice of 3 or 7 Site Skin fold Analysis
  Specifies which sites to use for 3-Site Skin fold pending men or women
  Calculates body fat results from standard protocol Skin fold
  measurements
  Provides Body Composition percentile of health status
  Calculates WHR
  Determines results of WHR
Upper Body Strength
  Configures whether sub-max or max test was performed
  Projects estimated max when sub-max test is performed
  Provides Upper Body Strength percentile
Lower Body Strength
  Configures whether sub-max or max test was performed
  Projects estimated max when sub-max test is performed
  Provides Lower Body Strength percentile
Cardiovascular (VO_{2MAX}) Power
  Configures whether sub-max or max test was performed
  Determines which test was performed based on information cued
  Calculates VO₂ for either sub-max or max pending on test performed
  Calculates non-exercise estimated VO₂
  Provides non-exercise estimated VO₂ results for comparison
  Provides Cardiovascular VO₂ Percentile
  Calculates Target Heart Rate Range
  Calculates MET Level Range
  Transposes Resting Heart Rate in beats per minute
  Transposes Blood Pressure when applicable
Flexibility
  Determines maximal effort from up to three readings
  Provides Flexibility percentile
```

Additional information that is preferably input to the device, either directly via user interfaces having prompts and/or other graphics for facilitating input, includes but is not limited to:

General Information, including but not limited to at least one client name, at least one test date, and corresponding client age, weight, sex, height is transposed;

Personal Goals, such as a listing of at least one client's own goals and their workout commitment regarding number of days per week, number of hours per day and current activity level; and Personal Trainer's name.

TABLE 2
Formulas

```
BMI = (weight * .454)/(height * .0254)²
Estimated % Fat from BMI:
      Males = (1.6 * BMI) − 25.1  Females = (2.2 * BMI) − 21.4
Men 3-Site Skinfold Measurements = chest/2 + abdomin/2 + thigh/2
Women 3-Site Skinfold Measurements = tricep/2 + suprailliac/2 + thigh/2
7-Site Skinfold Measurements =
    chest/2 + axilla/2 + tricep/2 + subscapular/2 + abdomin/2 + suprailliac/2 + thigh/2
Men 3-Site Body Density = 1.10938 − .0008267 (sum) + .0000016 (sum)² − .0002574 (age)
Women 3-Site Body Density = 1.099421 − .0009929 (sum) + .0000023 (sum)² − .0001392 (age)
Men 7-Site Body Density = 1.112 − .00043499 (sum) + .00000055 (sum)² − .00028826 (age)
Women 7-Site Body Density = 1.097 − .00046971 (sum) + .00000056 (sum)² − .00012828 (age)
Percentage Body Fat by Race and Sex:
    Indian (F) = (4.81/Db) − 4.34 * 100
    Indian (M) = (4.95/Db) − 4.50 * 100
    Black (F) = (4.85/Db) − 4.39 * 100
    Black (M) = (4.37/Db) − 3.93 * 100
    Hispanic (F) = (4.87/Db) − 4.41 * 100
    Hispanic (M) = (4.95/Db) − 4.50 * 100
    Asian (F) = (4.76/Db) − 4.28 * 100
    Asian (M) = (4.97/Db) − 4.52 * 100
    White (F) = (5.10/Db) − 4.66 * 100
    White (M) = (5.07/Db) − 4.64 * 100
    White (F) = (5.05/Db) − 4.62 * 100
    White (M) = (4.99/Db) − 4.55 * 100
    White (F) = (5.01/Db) − 4.57 * 100
    White (M) = (4.95/Db) − 4.50 * 100
Waist-To-Hip Ratio = Waist (in)/Hip (in)
Sub-Max Upper Body Strength = # Reps (2 to 15) ≈ 100% − (5% to 40%)
Estimated Upper Body Strength Max = Weight Pushed/Body Weight
Upper Body Strength Max = Weight Pushed/Body Weight
Sub-Max Lower Body Strength = # Reps (2 to 15) ≈ 100% − (5% to 40%)
Estimated Lower Body Strength Max = Weight Pushed/Body Weight
Lower Body Strength Max = Weight Pushed/Body Weight
Flexibility Max = Maximal number obtained
Rockport 1-Mile Walk Test = 132.853 − .1692 (weight kg) − .3877 (age) + 6.315 (men only) − 3.2649
(time min) − .1565 (end HR)
Bruce Treadmill Test = 14.8 − 1.379 (time min) + .451 (time min)² − .012 (time min)³
Houston Non-Exercise (M) = 67.350 − BMI (.754) − age (.381) + activity code (1.921)
Houston Non-Exercise (F) = 56.363 − BMI (.754) − age (.381) + activity code (1.921)
Queens College Step Test (M) = 111.33 − (.42 * HR end)
Queens College Step Test (F) = 65.81 − (.1847 * HR end)
Storer Cycle Ergometer Test = 403.4 + 10.22 (Mwatt) + 7.15 (wt kg) − 7.91 (age) − 252.2 (females
only)
Ebbeling 4-Minute Treadmill Walk Test = 15.1 + 21.8 (speed) − .327 (HR) − .263 (age * speed) + .00504
(HR * speed) + 5.980 (males only)
YMCA Cycle Test = 1.8 * Mwatt from chart/(wt kg) + 7 (220 − age) − HR
Target Heart Rate Range = 220 − age − RHR * (.5 & .75) + RHR
Target MET Level Range = VO₂/3.5 * (.5 & .75) + 3.5
Weight in kg = weight lbs * .454  Height in in = feet * 12 + inches
BMI status = range from <18.5 to >30
WHR status = Men range from >.94; Women range from >.82
Decision based formulas are based on information inputted from tests performed i.e.
choice 3 or 7-Site Skinfold, Max or Sub-Max Strength Test, VO₂ Test
Percentiles are obtained from applicable charts utilizing appropriate figures and statistics.
```

Additional formulas are provided in the attached documents (5 pages).

The basis of the present invention allows time efficiency for achieving results from a fitness evaluation and testing for both the client and trainer immediately after the testing is completed. The present invention defines the absolute fitness evaluation with ease by providing pertinent information about a client's fitness level by performing necessary calculated results from measurements and comparison figures as well as providing an average statistical percentile. The present invention revolutionizes a complete form of fitness evaluation by illustrates the highest standard in professionalism and technology in the fitness industry for both the client and personal trainer. Providing results is the foundation between the client and the personal trainer. The present invention bridges the initiation process providing a baseline to the client, which acts as a testable measure to illustrate progressive results throughout the transformation process. The transformation process provided by a personal trainer should be one that is measurable. The present invention allows accurate calculations throughout the client/trainer's relationship ensuring that measurable progressive results are being achieved through the services of a personal trainer.

A design example is shown in the attached figures, illustrating some graphic user interfaces of the device, including user inputs, and select, predetermined factors relevant for an individual's fitness evaluation.

Attached is a presentation of graphic user interfaces (GUIs) that are included in the present invention. A prompt or indication of what the user is to enter is provided, along with an example of an entry by the user, e.g., enter weight, enter height, body type, etc. An indication of other user entries based upon test results is also included in the present invention, by way of example and not limitation, percent body fat, blood pressure, resting heart rate, body measurements such as waist and hip, skin fold testing, and the like. Additionally, some GUIs provide for the user to make a selection from a predetermined listing of choices, e.g., male/female, race, etc. Depending upon which selection is made, a different series of GUIs may follow that correspond to that particular selection, such as which skin fold location is used. In some cases, a multiplicity of measurements are required; where a significant difference exists between the measurements for a single location, e.g., for skin fold, then an additional measurement may be prompted for and/or either a single reading is retained by the device or an average is taken and retained by the device. Furthermore, select GUIs indicate tests to be performed by an individual and then those test results are entered, by way of example and not limitation, upper body strength test, lower body strength test, cardiovascular tests, flexibility, etc. Note that each test may have a multiplicity of entries, including number of repetitions, time or duration of the test or exercise, weights used, and the like. An entry of personal goals and/or commitment level is also provided for in a GUI. Additional information like the personal trainer's name, number of evaluation, and the like. The device preferably provides for a storage capacity of at least one user, preferably a multiplicity of users, more particularly at least 50 users. Estimated body fat percentage loss and optimal weight range estimations are preferably provided for each user, based upon the information previously entered as set forth in the foregoing. Graphical output of information and/or results, and predicted performance gains and weight/body fat percentage loss is preferably provided by the present invention.

A method is also provided according to the present invention for automatic calculation of select, predetermined factors relevant for an individual's fitness evaluation and testing including the steps of providing a hand-held device having a software program with formulas relevant for an individual's fitness evaluation and testing; a user inputting data relevant to the individual to be evaluated; the device automatically calculating select, predetermined factors; and the device outputting the factors in a readable and/or printable format. According to the present invention, the method of using the hand-held device and formulas set forth hereinabove includes the steps of: providing a hand-held computer-type device used to store the formulas in an executable format when data is input by the user for the automatic calculation of select, predetermined factors relevant for an individual's fitness evaluation and testing; inputting information into the device;

differentiating between various information inputted during questionnaire that is pertinent in calculating proper equations based on specific tests performed but not limited to baseline data and test evaluation results;

determining proper calculation of specific equations regarding tests aid to determine and develop client's results;

providing explanations for test results in both common and scientific language;

determining results based on data entered and obtained from client's fitness evaluation/test through calculations from equations and data base information;

transposing select information from data entry to be included in the evaluation;

applying fitness evaluation results to resistance training and cardiovascular training programs as well as fitness evaluation test results;

describing safe and proper testing protocols as well as functioning for effective exercises during resistance and cardio training;

comparing between non-exercise/test results with actual accurate measurements performed during evaluation;

determining resting heart rate, training heart rate, and blood pressure with ease to client;

transporting and recording data throughout facility while testing of client is in progress;

maintaining database of clients' records from evaluation to evaluation;

producing graphing capabilities to illustrate progress with clients from evaluation to evaluation and workout to workout;

producing printable hardcopies for clients' personal files;

functioning in conjunction with and/or communicating with facilities' computer systems makes it fully operational without major upgrades.

Certain modifications and improvements will occur to those skilled in the art upon a reading of the foregoing description. By way of example, mobile telephones having expanded capabilities such as the capacity to handle spreadsheets or other software for formulaic calculations, internet access, and the like may be used in place of a hand-held computer-type device as described in the foregoing. Also, rather than having direct printing capabilities, the device as described may use a wireless connection to transmit data to a printer or other computer media for printing. All modifications and improvements have been deleted herein for the sake of conciseness and readability but are properly within the scope of the present invention.

What is claimed is:

1. A hand-held device for use in personal training and fitness evaluation comprising:
a portable device including a memory, a microprocessor, a power source, input/output interface for a user to input data and view outputs including results from processing the input data according to predetermined formulas relating to personal training and fitness for at least one individual;
software capable of running on the device for automatically calculating predetermined, select formulas associated with factors relevant to at least one individual's physical fitness.

2. The device of claim 1, wherein the device is capable of automatically generating the outputs.

3. The device of claim 1, wherein the outputs are capable of being provided in a printable format for providing a hard copy.

4. The device of claim 1, wherein the software includes the formulas in an executable format when data is input by the user for the automatic calculation of select, predetermined factors relevant for an individual's fitness evaluation and testing.

5. The device of claim 1, wherein the outputs are diagnostic physical fitness indicators.

6. A method for use in personal training and fitness evaluation comprising the steps of:
providing a handheld device of claim 1;
the user inputting data relating to an individual person;
the device providing outputs including predetermined factors relevant for an individual's fitness evaluation and testing.

7. The method of claim 6, further including the step of the device providing automatic calculation of the predetermined factors as outputs relevant for an individual's fitness evaluation and testing.

8. The method of claim 6, further including the step of the device outputting the factors in a readable and/or printable format.

9. The method of claim 6, further including the step of the user viewing the outputs on a user interface.

10. The method of claim 6, further including the step of storing the outputs in an electronic memory or database.

11. The method of claim 10, wherein the stored outputs are capable of being stored on the device or on removable computer readable medium.

12. The method of claim 6, further including the step of the user comparing the outputs to prior output information relating to the same individual person being evaluated.

13. The method of claim 6, wherein the device is a hand-held computer-type device used to store the formulas in an executable format when data is input by the user for the automatic calculation of select, predetermined factors relevant for an individual's fitness evaluation and testing.

14. The method of claim 6, including a step of inputting information into the device during a physical fitness diagnostic testing, training, or personal training session.

15. The method of claim 6, further including a step of the device automatically differentiating between various information inputted during questionnaire that is pertinent in calculating proper equations based on specific tests performed but not limited to baseline data and test evaluation results.

16. The method of claim 6, further including a step of the device automatically determining proper calculation of specific equations regarding tests required to aid, determine, and develop improved results for the individual.

17. The method of claim 6, further including a step of providing explanations for test results in both common and scientific language.

18. The method of claim 6, further including a step of determining results based on an integrated data set including but not limited to input data entered and obtained from client's fitness evaluation in real-time.

19. The method of claim 18, wherein the integrated data set further includes prior outputs from equations and/or historical database information.

20. The method of claim 6, further including a step of applying fitness evaluation results to resistance training and cardiovascular training programs as well as fitness evaluation test results.

21. The method of claim 6, further including a step of describing safe and proper testing protocols and/or effective exercises to be performed during resistance and cardio training for effecting improvements based upon the outputs and individual's goals information input.

22. The method of claim 6, further including a step of comparing between non-exercise/test results with actual measurements performed during evaluation, the comparison being made automatically by the device and viewable by the user.

23. The method of claim 6, further including a step of determining resting heart rate, training heart rate, and blood pressure for the individual being evaluated by the user.

24. The method of claim 6, further including a step of transporting and recording data throughout a workout location or facility while testing of the individual is in progress by the user using the device.

25. The method of claim 6, further including a step of maintaining a database of individual user records by individuals evaluated.

26. The method of claim 6, further including a step of producing graphical outputs on a graphical user interface of the device to illustrate progress with individuals over time.

27. The method of claim 26, wherein the progress is considered from a preceding evaluation to a subsequent evaluation and/or a preceding workout to a subsequent workout.

28. The method of claim 26, wherein the progress is capable of being considered over a predetermined time period, the time period being selectable by the user and indicated by user inputs to the device.

29. The method of claim 6, further including a step of producing tangible outputs in computer readable medium or printable hardcopies format.

30. The method of claim 6, further including a step of functioning in conjunction with and/or communicating with other computerized equipment for exercise and/or testing, computers and/or networked computers computer systems.

31. A hand-held computer-type device for use in personal training comprising:
a hand-held computer having memory, a microprocessor, a power supply, a user interface having input/output capability;
formulas in an executable format stored on the device;
input data provided by a user relating to an individual;
the input data being processed by the formulas to produce output factors viewable on the user interface, the factors being relevant for an individual's fitness evaluation and testing.
